(19) United States
(12) Patent Application Publication
(10) Pub. No.: US 2009/0005652 A1
(43) Pub. Date: Jan. 1, 2009

(54) METHOD AND SYSTEM FOR PERMITTING ACCESS TO EQUIPMENT, DEVICES, SYSTEMS, SERVICES OR THE LIKE BASED ON SLEEP QUALITY ANALYSIS

(76) Inventor: Ron Kurtz, Oakville (CA)

Correspondence Address: BERESKIN AND PARR, 40 KING STREET WEST, BOX 401, TORONTO, ON, M5H 3Y2, CA

(21) Appl. No.: 12/115,744

(22) Filed: May 6, 2008

Related U.S. Application Data
(60) Provisional application No. 60/916,429, filed on May 7, 2007.

(51) Int. Cl. A61B 5/00

(52) U.S. Cl. 600/300

(57) ABSTRACT

Methods and systems for controlling a subject's access to an activity based on a sleep quality index are described. The sleep quality index may related to various physiological data relating to the subject, including current and historical physiological data, previous sleep quality indices for the subject. The subject's access to the activity may also be restricted based on personal characteristics of the subjects or on the identity of the subject.

This is an application claiming the benefit under 35 USC 119(e) of U.S. Provisional Patent Application Ser. No. 60/916,429 filed May 7, 2007. U.S. Ser. No. 60/916,429 is incorporated herein, in its entirety, by this reference to it.

### FIELD

The described embodiments relate to methods, systems and devices for permitting or restricting access to equipment, devices, systems, services and other items based on sleep quality analysis. In particular, embodiments involve determining a sleep quality index and permitting or restricting access based on the sleep quality index.

### BACKGROUND

There are many activities that people engage in that may be impaired by insufficient or low quality sleep. Depending on the activity, the person performing the activity may be more likely to perform the activity in a dangerous or inadequate manner if the person's performance is impaired by low quality sleep. Such activities may include, but are not limited to, driving, piloting a plane, operating heavy machinery, and performing surgery.

Low quality sleep may be caused by a variety of reasons including sleep disorders such as obstructive sleep apnea (OSA). According to the American National Institute of Heath, OSA currently affects more than twelve million Americans (4% of men and 2% of women), making this disorder as common as adult diabetes. Such sleep disorders may be responsible for job impairment and motor vehicle crashes. For example, the Department of Transport in the UK estimates that 20% of road accidents leading to death and serious injury are caused by drowsiness or sleep disorders.

Thus, it is desirable that people who have not received a proper quality of sleep be prevented from engaging in certain activities in which harm may come to themselves or others.

### SUMMARY

In one aspect the present invention provides a method of restricting an activity based on sleep quality, said method comprising: monitoring physiological data of a subject; determining at least one sleep quality index based on the physiological data; determining whether to permit the activity based on the at least one sleep quality index.

In some embodiments, the physiological data is selected from the group consisting of breathing patterns, electroencephalographic signals, electromyographic signals, electrooculographic signals, blood oxygen levels, body position, body movement, heart rate, arterial tone, plethismography, and snoring levels.

In some embodiments, the method further includes determining at least one sleep quality index based on current physiological data, wherein the current physiological data comprises physiological data obtained from a current sleep session.

In some embodiments, the method includes storing the physiological data; and determining at least one sleep quality index based on historical physiological data, wherein the historical physiological data comprises physiological data stored from a previous sleep session.

In some embodiments, the method further includes determining at least one sleep quality index based on current physiological data and historical physiological data.

In some embodiments, the method further includes storing the at least one sleep quality index.

In some embodiments, the method further includes determining at least one sleep quality index based on current physiological data and the at least one stored sleep quality index.

In some embodiments, at least one sleep quality index is determined based on at least one personal characteristic of the subject.

In some embodiments, the at least one personal characteristic comprises a sleep time.

In some embodiments, at least one sleep quality index is determined based on at least one characteristic of the activity.

In some embodiments, the determination of whether to permit access is based on the sleep index.

In some embodiments, if it is determined that the activity should not be permitted, then restricting access to a restricted access area; and if it is determined that the activity should be permitted, then permitting access to the restricted access area.

In some embodiments, the method further includes: verifying the identity of the subject; if it is determined that the activity should not be permitted, then restricting access to a restricted access area; and if it is determined that the activity should be permitted and the identity of the subject has been verified, then permitting access to the restricted access area.

In some embodiments, the method further includes permitting and restricting access is accomplished by locking and unlocking a door.

In some embodiments, the method further includes: if it is determined that the activity should not be permitted, then restricting access to a restricted access equipment; and if it is determined that the activity should be permitted, then permitting access to the restricted access equipment.

In some embodiments, the method further includes the method further includes: verifying the identity of the subject; if it is determined that the activity should not be permitted, then restricting access to a restricted access equipment; and if it is determined that the activity should be permitted and the identity of the subject has been verified, then permitting access to the restricted access equipment.

Another aspect of the invention provides a system for restricting an activity based on sleep quality, the system comprising: monitoring device for monitoring physiological data of a subject; a sleep assessment device for determining at least one sleep quality index based on the physiological data; and an access device for determining whether to permit the activity based on the at least one sleep quality index.

In some embodiments, the monitoring device is operable to monitor physiological data wherein the physiological data is selected from the group consisting of breathing patterns, electroencephalographic signals, electromyographic signals, electrooculographic signals, blood oxygen levels, body position, body movement, heart rate, arterial tone, plethismography, and snoring levels.

In some embodiments, the sleep assessment device is operable to determine at least one sleep quality index based on current physiological data, wherein the current physiological data comprises physiological data obtained from a current sleep session.

In some embodiments, the sleep assessment device comprises a memory for storing the physiological data and is operable to determine at least one sleep quality index based on historical physiological data, wherein the historical physiological data comprises physiological data stored from a previous sleep session.

In some embodiments, the sleep assessment device is operable to determine at least one sleep quality index based on current physiological data and historical physiological data.

In some embodiments, the sleep assessment device comprises a memory for storing the at least one sleep quality index.

In some embodiments, the sleep assessment device is operable to determine at least one sleep quality index based on current physiological data and the at least one stored sleep quality index.

In some embodiments, the sleep monitoring device comprises a memory for storing the physiological data; and

the sleep assessment device is operable to determine at least one sleep quality index based on historical physiological data, wherein the historical physiological data comprises physiological data stored from a previous sleep session.

In some embodiments, the sleep assessment device is operable to determine at least one sleep quality index based on current physiological data and historical physiological data.

In some embodiments, the sleep assessment device is operable to determine at least one sleep quality index based on at least one personal characteristic of the subject.

In some embodiments, the at least one personal characteristic comprises a sleep time.

In some embodiments, the sleep assessment device adjusts the at least one sleep quality index based on at least one characteristic of the activity.

In some embodiments, the access device determines whether to permit access based on the sleep quality index.

In some embodiments, the access device restricts access to a restricted access area if it is determined that the activity should not be permitted; and the access device permits access to a restricted access area if it is determined that the activity should be permitted.

In some embodiments, the access device verifies the identity of the subject; the access device restricts access to a restricted access area if it is determined that the activity should not be permitted; and the access device permits access to a restricted access area if it is determined that the activity should be permitted and the identity of the subject has been verified.

In some embodiments, the access device permits and restricts access by locking and unlocking a door.

In some embodiments, the access device restricts access to a restricted access equipment if it is determined that the activity should not be permitted; and the access device permits access to a restricted access equipment if it is determined that the activity should be permitted.

In some embodiments, the access device verifies the identity of the subject; the access device restricts access to a restricted access equipment if it is determined that the activity should not be permitted; and the access device permits access to a restricted access equipment if it is determined that the activity should be permitted and the identity of the subject has been verified.

In some embodiments, the system further includes an identification device for interfacing with the access device.

In some embodiments, the identification device has a memory for storing the physiological data.

In some embodiments, the identification device has a memory for storing the at least one sleep quality index.

The system as defined in claim 35, wherein the identification device is selected from a group consisting of a key, a device incorporating a RFID tag, a smart card or other memory device.

Additional features of the present invention are also described below in the context of a detailed description of various exemplary embodiments.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a block diagram of a sleep quality access system according to various embodiments;

FIG. 2 is a flowchart of an example set of basic operational steps executed by the sleep quality access system of FIG. 1;

FIG. 3 is a block diagram of the identification device of FIG. 1; and

FIG. 4 is a block diagram of the access device of FIG. 1.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

It will be appreciated that for simplicity and clarity of illustration, where considered appropriate, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

The embodiments of the systems and methods described herein may be implemented in hardware or software, or a combination of both. For example, some embodiments are implemented in computer programs executing on programmable computers each comprising at least one processor, a data storage system (including volatile and non-volatile memory and/or storage elements), at least one input device, and at least one output device. Other embodiments may be implement using programmable computer devices such as programmable logic arrays, programmable logic controllers, microcontrollers, microcomputers, personal computers, laptop computers, personal data assistants, and cellular telephones. Program code is applied to input data to perform the functions described herein and generate output information. The output information is applied to one or more output devices, in known fashion.

Each program is preferably implemented in a high level procedural or object oriented programming and/or scripting language to communicate with a computer system. However, the programs can be implemented in assembly or machine language, if desired. In any case, the language may be a compiled or interpreted language. Each such computer program is preferably stored on a storage media or a device (e.g. ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

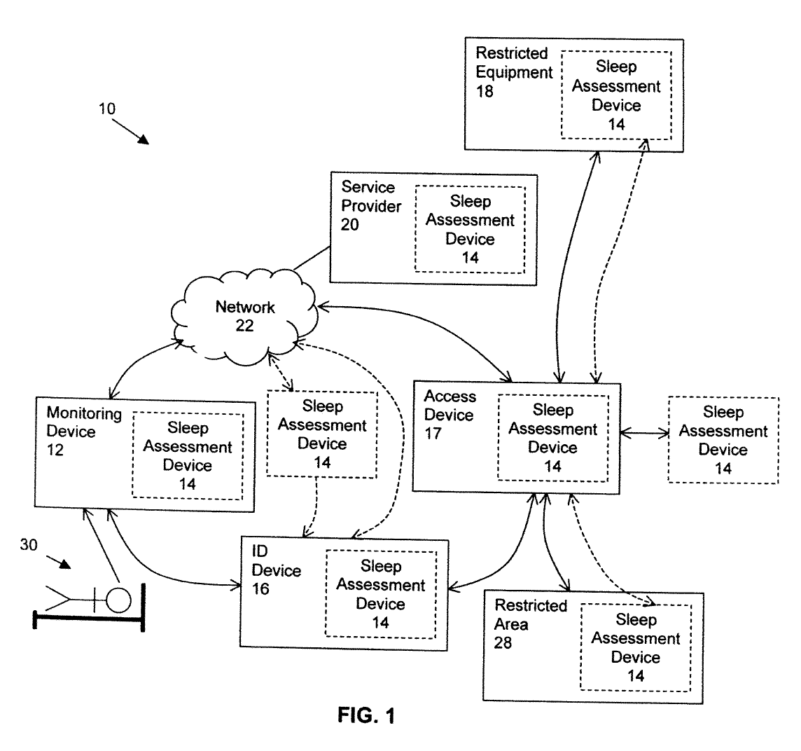

FIG. 1 illustrates an exemplary embodiment of a sleep quality access system 10. Specifically, sleep quality access system 10 includes a sleep monitoring device 12, a sleep assessment device 14, and an identification (ID) device 16, with restricted access equipment 18 and/or a restricted access area 28, a service provider system 20, and a network 22.

As discussed in more detail above, it should be understood that sleep quality access system 10 may be implemented in hardware or software or a combination of both. In the present embodiment, the modules of sleep quality access system 10 are implemented in computer programs executing on programmable computers each comprising at least one processor, a data storage system and at least one input and at least one output device. Without limitation the sleep quality access system 10 may be implemented in any type of computing device, such a programmable logic array, a microcontroller, a microprocessor, a mainframe computer, server, personal computer, laptop, personal data assistant or cellular telephone.

Sleep quality access system 10 is used to monitor subject 30's quality of sleep and determine whether or not to permit subject 30 to engage in one or more activities, which may be referred to as restricted activities. In various embodiments, this is accomplished by restricting the subject's access to certain restricted access equipment 18 and/or restricted access areas 28 based on the quality of sleep experienced by subject 30 prior to attempting to access restricted access equipment 18 or restricted access area 28.

More specifically, in various embodiments, monitoring device 12 is used to monitor physiological data. Sleep assessment device 14, is utilized to assess the quality of sleep based on the physiological data and to produce a sleep quality index that is reflective of the quality of sleep. In some embodiments, sleep quality access system 10 utilizes ID device 16 to identify subject 30. In addition, in some embodiments, ID device 16 may be utilized to transport data, such as physiological data and sleep quality indices. Access device 17 is utilized to permit or deny subject 30 access to restricted access equipment 18 or restricted access area 28. In various embodiments, sleep access system 10 may provide a third party, such as a service provider, which may be, but is not limited to, a medical doctor, with the subject's physiological data for treatment and monitoring purposes.

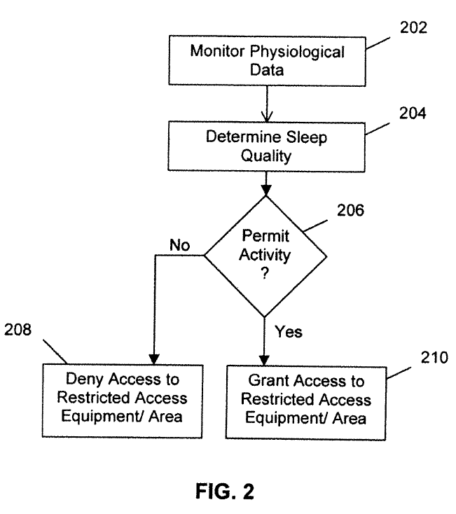

Reference is next made to FIG. 2, which is a flowchart of an example set of basic operational steps 200 executed by various embodiments of sleep quality access system 10. The process starts at step 202, where subject 30's physiological data is measured by monitoring device 12. As will be discussed in greater detail below, monitoring device 12 may measure any appropriate physiological data which may be used to determine the quality of sleep of subject 30.

At step 204, sleep assessment device 14, determines the quality of subject 30's sleep based on the physiological data obtained at step 202. As will be discussed in greater detail below, sleep assessment device 14 may produce one or more sleep quality indices that are indicative of the sleep quality received by subject 30. In various embodiments, the factors that may be taken into account when producing the sleep quality indices may include, but are not limited to, personal characteristics of subject 30, present and historical physiological data obtained from subject 30, and characteristics of the restricted activity.

At step 206, it is determined whether subject 30 should be permitted to perform the restricted activity. In various embodiments, this determination is made based on the sleep quality that subject 30 was determined to have experienced at step 204. If it is determined that subject 30 should not be permitted to perform the restricted activity, then step 208 is executed. On the other hand, if it is determined that subject 30 should be permitted to perform the restricted activity then step 210 is executed.

At step 208, access device 17 denies subject 30 access to the restricted access equipment 18 or restricted access area 28. This may be performed in any appropriate manner, which will be discussed in further detail below. For example, access device 17 may prevent a door, which may lead to restricted access area 28, from unlocking or opening. Similarly, access device 17 may lockdown the controls of restricted access equipment 18.

At step 210, access device 17 permits subject 30 to have access to the restricted access equipment 18 or restricted access area 28. This may be performed in any appropriate manner, which will be discussed in further detail below. For example, access device 17 may unlock or open a door that leads to restricted access area 28. Similarly, access device 17 may unlock the controls of restricted access equipment 18.

The term “quality of sleep”, may refer to a wide variety of characteristics of sleep including, but not limited to, the total length of sleep, the longest length of unbroken sleep, the number of interruptions to the sleep, the length of rapid eye movement (REM) sleep, the length of non-rapid eye movement (NREM) sleep, the amount of time since the last sleep session, the amount of snoring, the average heart rate, the number or rate of respiratory events, the intensity of each respiratory event, the number or rate of arousals, the duration of each arousal, and the intensity of each arousal, the portion of the time in bed spent asleep, the time taken to fall asleep, the length or portion of time spent in deep sleep, and the number or rate of oxygen desaturations. The quality of sleep may also be defined as a mathematical or rule based expression derived from a combination of the above characteristics.

In some embodiments, the term “quality of sleep” may characterize the sleep experienced by subject 30 in the most recent sleep session. In other embodiments, quality of sleep may also characterize the sleep experienced by subject 30 over a number of sleep sessions. Thus, for example, if subject 30 sleeps every night for n days in a row, one may refer to the quality of sleep that subject 30 experienced in each of the individual nights. One may also refer to the quality of sleep that subject 30 experienced over the period of n days. Thus, the term quality of sleep may also refer to a cumulative characterization of the sleep that subject 30 experienced over a given time period.

Subject 30 may engage in an activity that could directly or indirectly cause or allow harm or injury to occur to themselves and others, which may include but is not limited to, damage to property, bodily harm or injury, psychological harm, death, and financial harm. The restricted activity may be for example, but is not limited to, a job performed by subject 30 or a task performed by subject 30 in the course of his/her employment. For example, but not limited to, subject 30 may be an air traffic controller, a driver, a pilot, a locomotive engineer, or a surgeon.

Various people, such as for example people who engage in the above listed occupations, may perform tasks that may lead to harm if the person performing them did not experience a certain quality of sleep. Specifically, when a person's sleep quality is compromised, his or her performance may be affected in a myriad of ways including but not limited to his/her alertness, reaction time, memory, ability to reason, and coordination. Thus, if subject 30 were to operate equipment, such as a vehicle, after not experiencing the required quality of sleep he or she may injure someone by not reacting in time or by dosing off while operating the equipment. Similarly, a surgeon that has not had the proper quality of sleep may cause the death of a patient due to lack of coordination.

A restricted activity may involve subject 30 operating restricted access equipment 18 through which he/she may cause harm if he/she has not had a required quality of sleep. The restricted access equipment 18 may include for example, but is not limited to, vehicles, heavy machinery, dangerous tools, explosives and weapons. Thus, restricted access equipment 18 may include, but is not limited to, trucks, cars, forklifts, trains, buses, ships, airplanes, cranes, drills, leveling equipment, and excavation equipment. Subject 30 may be prevented from operating restricted access equipment 18 if sleep access system 10 detects a sleep quality below a certain threshold. This may be done in any appropriate manner, including but not limited to locking down controls or disabling an ignition switch.

Alternatively, a restricted activity may in some way require that subject 30 have access to a restricted access area 28. For example, subject 30 may need to operate in or fulfill a role in restricted access area 28. Alternatively, subject 30 may need access to equipment that is stored in restricted access area 28. Further still, subject 30 may need to pass through restricted access area 28. Thus, restricted access area 28 may be, but is not limited to, an air traffic control room, an operating room, or a locker in which equipment is stored. Subject 30 may be prevented from entering restricted access area 28 if sleep access system 10 detects a sleep quality below a certain threshold.

It should be understood that in various embodiments, there may be some overlap between the restricted access equipment 18 and restricted access area 28. In particular, restricted access area 28 may be an area within restricted access equipment 18. For example, restricted access area 28 could be a cockpit in an airplane, while the airplane could be restricted access equipment 18. Alternatively, restricted access equipment 18 could be located within a restricted access area 28. For example, restricted access equipment 18 could be the computer systems in an air traffic control room, which itself could be the restricted access area 28.

In some embodiments, sleep access system 10 may restrict access to both a restricted access area 28 and restricted access equipment 18. For example, sleep access system 10 may prevent subject 30 from entering an air traffic control room and may additionally lock down subject's 30's computer workstation in the air traffic control room.

Monitoring device 12 may monitor and record data that may be used to determine the quality of sleep of subject 30. For example, monitoring device may monitor physiological data, which may include but is not limited to, breathing patterns, electroencephalographic (EEG) signals, electromyographic (EMG) signals, electrooculographic (EOG) signals, blood oxygen levels, body position, body movement, heart rate, arterial tone, and snoring levels. In various embodiments, some of these signals may be measured through the use of electrodes that may be placed according to standard electrode placement techniques. In addition, the electrodes may be applied according to techniques described in U.S. patent application Ser. Nos. 11/131,284 and 11/615,584, which are incorporated herein by reference.

In various embodiments, monitoring device 12 may include a means of providing positive air pressure in order to treat or prevent sleep apnea. In some embodiments, the positive air pressure may be applied in a continuous manner such as in a CPAP device.

In some embodiments, monitoring device 12 may also include a means of verifying the identity of the person whose sleep is being monitored. This may be done to ensure that the physiological data is that of subject 30 and not of someone else. This may be accomplished in any appropriate manner, such as biometric measures of fingerprints or EEG patterns.

In various embodiments, monitoring device may include a memory for storing physiological data. The term “current physiologic data”, as used herein, refers to physiological data from the most recent sleep session experienced by subject 30. The most recent sleep session may be referred to as the current sleep session. The term “historical physiologic data”, as used herein, refers to physiological data from sleep sessions prior to the most recent sleep session.

Sleep assessment device 14 may use data that is monitored and recorded by monitoring device 12 in order to determine a quality of sleep of subject 30. In particular, in various embodiments, sleep assessment device 14 may compute one or more indices that are representative of the quality of sleep of subject 30. In addition, sleep assessment device 14 may store historical data, such as historical physiological data or historical sleep quality indices, of the quality of sleep of subject 30. Sleep assessment device 14, may use such historical sleep quality data in determining one or more of the sleep quality indices. In this manner, at least one of the indices may reflect a cumulative quality of sleep that accounts for the historical quality of sleep for subject 30.

In some embodiments, each of the sleep indices produced by sleep assessment device 14 may be adjusted according to personal characteristics of subject 30, which may include, but are not limited to, the amount of sleep normally required by subject 30, and subject age, medications. Moreover, in various embodiments, each of the sleep indices produced by sleep assessment device 14 may be adjusted according to the activity performed by subject 30. For example, certain activities may demand that a particular subject 30 have a greater quality of sleep than other activities.

A sleep index, or sleep quality index (SQI), may be calculated in various ways. In some embodiments, a sleep quality index is calculated according to a formula:

SQI=f(Pᵢ),

where: Pᵢ represents a set of i parameters; and
f(Pi) represents a function applied to the parameters Pi to determine the sleep quality index.
   Various parameters may be incorporated in the calculation of a sleep quality index. For example, the list of parameters may include some or all of the following parameters:

Sleep time (ST, the time spent asleep);

Sleep efficiency (SE, the ratio of sleep time over time in bed);

Sleep latency (SL, the time to fall asleep);

REM sleep latency (REMSL, the time to enter REM sleep);

Duration of time in stage wake (DurW);

Duration of time in stage REM (DurREM);

Duration of time in stage 1 (Dur1);

Duration of time in stage 2 (Dur2);

Duration of time in stage deep sleep (DurDS);

Number of wakenings during sleep time (NW);

Number of arousals during sleep time (NA);

Wakening index (WI, number of wakenings per hour of sleep);

Arousal index (AI, number of arousals per hour of sleep)

Age;

Sex; and

Medical History.

Various parameters may have a greater or lesser effect on quality of sleep. Some factors may have a proportional relationship with quality of sleep while others have an inverse relationship. For example, in some embodiments, various parameters may be assigned different weights and the weighted parameters may be combined together to provide a sleep quality index:

$$SQI = {\sum }_{i = 1}^{n} {{W}_{i} ( {P}_{i} )}^{{Q}_{i}} ,$$

where: n is the number of parameters;
Pᵢ is a parameter from the above set;
Wᵢ is the weighting coefficient; and
QᵢεE{1,−1} corresponds to direct or inverse proportionality.
   In other embodiments, the relationship of a particular parameter to the sleep index, or sleep quality index, may be any relationship and the sleep quality index may be calculated as follows:

$$SQI = {\sum }_{i = 1}^{n} {f}_{i} ( {P}_{i} )$$

where: fᵢ is a function specific to parameter Pᵢ.

The selection of parameters, specific weight assigned to any specific parameter or the function applied to any specific parameter may be determined in various manners. In some embodiments, a subjective sleep quality index may be determined based on sleep studies performed on a patient, which may include monitoring a patient during sleep and analysis of the patient's perception of their quality of sleep and parameters relevant to their quality of sleep. Parameters identified in such sleep studies may then be weighted using a standardized weighting or using a weighting specific to the patient. Patient specific weighting coefficients or functions may be determined, for example, using least-square-root (i.e. least square error), neural network or other methods.

In various embodiments, sleep assessment device 14 may also make a determination of whether subject 30 should be permitted to perform a certain activity. In such embodiments, sleep assessment device 14 may produce any appropriate sleep quality index, such as a binary sleep quality index, which in one state indicates that the subject 30 should be permitted to perform the activity and/or in the other state indicates that subject 30 should not be permitted to perform the activity.

In some embodiments, sleep assessment device 14 may produce multiple sleep quality indices when subject 30 may potentially perform multiple restricted activities. Each sleep quality index may correspond to a particular restricted activity, or a type of restricted activity, or a class of restricted activities.

In some embodiments, the sleep indices produced by sleep assessment device 14 may not take into account the activity performed by subject 30. In addition, in various embodiments sleep assessment device 14 does not make a decision with respect to whether subject 30 should be allowed to perform a restricted activity. In such embodiments, access device 17 may make the decision with respect to whether subject 30 should be allowed to perform the restricted activity. For example, different access devices 17 may correspond to different restricted activities. Each access device 17 that corresponds to a different restricted activity may have a different threshold for permitting subject 30 to perform the restricted activity based on the sleep quality index.

In various embodiments, sleep assessment device 14 may comprise any appropriate computing device. In some embodiments, sleep assessment device 14 may comprise, but is not limited to, a personal computer, a laptop, and a PDA.

In various other embodiments, sleep assessment device 14 may be a dedicated computing device that may be integrated with various other systems. For example, but not limited to, sleep assessment device 14 may be integrated with monitoring device 12, ID device 16, and access device 17.

In addition, in various embodiments sleep assessment device 14 may be implemented at various locations. For example, sleep assessment device 14 may be located at restricted access equipment 18 or restricted access area 28, at service provider 20, at subject 30's home, anywhere subject 30 may sleep, or anywhere subject 30 may carry a portable sleep assessment device 14. In some embodiments, there may be multiple sleep assessment devices 14.

In various embodiments, sleep access device 14 may be linked to other elements of sleep access system 10 through any appropriate communication link which may be for example, but is not limited to, a network, such as network 22, a direct wire line, an electromagnetic communication link, such as radio frequency link, microwave link, or fiber optic link.

Moreover, in various embodiments, sleep assessment device may not be directly connected to any other system. In some embodiments, ID device 16 may provide a means of transferring data between sleep assessment device 14 and other elements of sleep quality access system 10.

In some embodiments, sleep quality access system 10 utilizes ID device 16 to identify subject 30. In various embodiments, ID device 16 may be any device that may be used to identify a particular individual including, but not limited to, a key, any device incorporating a radio frequency identification (RFID) tag, a smart card, a chip card, an integrated circuits card, a swipe card, and a portable computing device, such as a PDA. For example, a subject 30 may be given a key, smart card, or swipe card, to which only he/she has access and therefore it may be used to identify him or her.

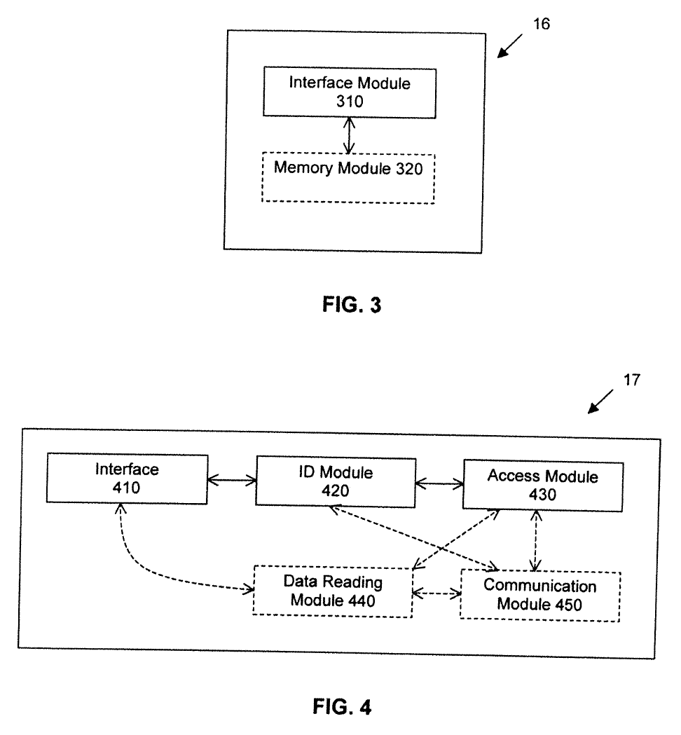

Reference is now made to FIG. 3, which is a block diagram of ID device 16. ID device 16 comprises an interface module 310. In addition, although not illustrated in FIG. 3, in some embodiments, ID device 16 may include sleep assessment device 14. Interface module is used to interface with other elements of sleep access system 10. For example, interface module 310 allows ID device 16 to interface with sleep access device 14. Interface module 310 may include, but is not limited to, the blade of a key, a magnetic strip of a swipe card, a RFID tag, or a chip of a smartcard. In some embodiments, interface module 310 allows ID device 16 to also interface with monitoring device 12 and sleep assessment device 14. In addition, in various embodiments, interface module 310 allows ID device 10 to download and upload data.

In some embodiments, ID device 16 may incorporate a memory module 320. Memory module 320 may be used to store any data that is transferred through interface module 310, including but not limited to raw physiological data as well as sleep quality indices. In some embodiments, memory module 320 may be part of interface module 310 such as in the case of a magnetic strip of a swipe card. Memory module 320, may be any appropriate medium for storing data including but not limited to, random access memory (RAM), a hard drive, the chip of a smartcard, the magnetic strip of a swipe card, an optical disk, and electronically programmable read only memory (EPROM).

For example, in some embodiments, memory module 320 allows ID device 16 to store physiological data provided by monitoring device 12. ID device 16 may interface with monitoring device 12 to download the physiological data after a sleep session. ID device 16 may then interface with sleep assessment device 14 and upload the physiological data to sleep assessment device 14. Sleep assessment device 14 may then determine sleep quality indices, which may then be downloaded to the memory module 320 of ID device 16. ID device 16 may then interface with access device 17 and download the sleep quality indices to access device 17.

Thus, the above described sleep quality indices, which are determined by sleep assessment device 14, may be provided to the access device 17 in any appropriate manner. This will be discussed in greater detail below.

In various other embodiments, sleep assessment device 14 may be integrated with monitoring device 12. In such embodiments, ID device 16 could be used to record data generated by sleep assessment device 14. For example, this data may include a decision of whether or not to grant the subject 30 access to the restricted access equipment 18 or restricted access area 28. In such embodiments, subject 30 may then take the ID device 16 to the restricted access equipment 18 or restricted access area 28 where the data can be read by access device 17 and access may be granted or denied based on the data stored on ID device 16.

In various other embodiments, an ID device 16 may not be used. In such embodiments, monitoring device 12, sleep assessment device 14, and access device 17, which may each be located at various locations, may be coupled together through a communication link such as network 22. All relevant physiological data may be transferred through the communication link. In such embodiments, access device 17 may have a different means of identifying subject 30, other than ID device 16, such as for example, biometric data. This will be discussed in greater detail below.

Reference is now made to FIG. 4, which is a block diagram of various embodiments of an access device 17, which may be used to restrict access to restricted access equipment 18 or restricted access area 28. Access device 17 comprises an interface 410, ID module 420, and access module 430. In various embodiments, access device 17 may also comprise a data-reading module 440. Moreover, some embodiments may also comprise a communication module 450. In addition, although not illustrated in FIG. 4, in some embodiments, access device 17 may include sleep assessment device 14.

In various embodiments, access device 17 may be coupled to a mechanism or system that could be used to prevent subject 30 from gaining access to the restricted access equipment 18 or restricted access area 28. In the case of restricted access equipment 18, access device 17 may be coupled to a variety of mechanisms or systems including, but not limited to, a control system for the restricted access equipment 18, an ignition system for restricted access equipment 18, a power connection for restricted access equipment 18, or a log on interface for restricted access equipment 18.

In the case of restricted access area 28, access device 17 may be coupled to a variety of mechanisms or systems including, but not limited to, a door leading to the restricted access area 28, a door to leading to a restricted access area 28 within restricted access equipment 18, such as a car door or a door to a crane, or a door to an area within the equipment, such as a door to the cockpit of a plane or a door to the bridge of a ship, a door to a room or locker that contains the restricted access equipment 18, or a door to a garage where the equipment is parked.

Thus, for example, in the case of an air traffic controller, access device 17 may be coupled to a door to the air traffic control room (restricted access area 28) and thereby prevent access to the actual room when subject 30 attempts to enter. Alternatively, access device 17 may be coupled to a system (restricted access equipment 18) used by subject 30 in fulfilling his or duties. Thus, access device 17 may disable the system and thereby prevent subject 30 from fulfilling his or duties. For example, access device 17 may prevent an air traffic controller from logging onto a computer system that he or she uses when directing air traffic. Alternatively, access device 17 may preclude access to both the restricted access area 28 and restricted access equipment 18.

Interface 410 may be any appropriate device for receiving any appropriate identifier of subject 30. Identifiers may include, but are not limited to, keys, smart cards, RFID tags, id cards, swipe cards, finger prints, palm prints, iris or retinal patterns, voice commands, codes, EEG fingerprinting, and facial features. Thus, interface 410 may comprise, but is not limited to, a keyhole, a swipe card reader, a keypad, a smart card reader, a biometric scanner, a fingerprint scanner, a camera, a microphone, and an iris scanner.

Identification module 420, may be any hardware or software, or combination of the two that may identify subject 30 based on the identifier received at the interface 410. Identification module 420 interoperates with interface 410 in order to identify the subject 30. Thus, identification module 420, may comprise, but is not limited to, a tumbler of a key lock, hardware and/or software that may identify subject 30 based on his/her biometric information such as his/her voice, fingerprints, palm prints, iris patterns, and facial features.

Data reading module 440, may be any appropriate software or hardware, or a combination of the two, that may read physiological data that is provided to interface 410 by for example an ID device 16. Thus, for example data reading module 440 may include, but is not limited to, any hardware or software that may interoperate with interface 410 to read the information stored on a smart card, the magnetic strip of a swipe card.

Access module 430 may be coupled to other systems that may prevent subject 30 from gaining access to restricted access area 28 or from operating restricted access equipment 18. Thus, for example, access module 430 may be coupled to a control system for a door or a lock of a door that leads to restricted access area 28. Similarly, access module 430 may be coupled to an ignition system of a vehicle that is the restricted access equipment 18. Moreover, access module 430 may be coupled to a power source for a workstation that may prevent power reaching the workstation if subject 30 has not had the required quality of sleep.

Communication module 450 may interface with other elements of sleep quality access system 10 through any appropriate means including but not limited to a network such as network 22 or a direct communication link whether through wire, fiber optic cable or radio frequency link.

In some embodiments, physiological data may be provided to access device 17 by ID device 16. In such embodiments, communication module 450 may receive physiological data from data reading module 440. In various embodiments, communication module 450 may also receive identification data from ID module 420, which could be used to identify the particular subject 30. Communication module 450 may then forward this data to sleep assessment device 14. Sleep assessment device 14 then processes the physiological data and any other data, such as identification data, to produce one or more sleep quality indices. Sleep assessment device 14 then transmits the one or more sleep quality indices to communication module 450. Communication module 450 then provides the one or more indices to access module 430. Access module 430 then permits or restricts access according to the sleep quality indices received.

In some embodiments, physiological data may not be provided to access device 17 by ID device 16. In such embodiments, communication module 450 may receive identification data from ID module 420. Based on the identification data communication module 450 may send a request to sleep assessment device 14 for one or more sleep quality indices for subject 30. Depending on the particular embodiment of sleep access system 10, sleep assessment device 14 may already have the raw physiological data or it may retrieve it from monitoring device 12 through any appropriate communication link such as network 22. Sleep assessment device 14 then determines any appropriate sleep quality indices. Sleep assessment device 14 then transmits the admittance data to communication module 450. Communication module 450 then provides this data to access module 430.

In some embodiments, ID device 16 may provide one or more sleep quality indices to access device 17. In such embodiments, the admittance data is read by data reading module 440 and provided to access module 430. In addition, identification data is provided by ID module 420 to access module 430. Access module 430 then permits or restricts access accordingly. In such embodiments, access device 17 may not include communication module 450.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto.

I claim:

1. A method of restricting an activity based on sleep quality, said method comprising:
monitoring physiological data of a subject;
determining a sleep quality index based on the physiological data; and
determining whether to permit the activity based on the sleep quality index.

2. The method of claim 1 wherein the physiological data is selected from the group consisting of breathing patterns, electroencephalographic signals, electromyographic signals, electrooculographic signals, blood oxygen levels, body position, body movement, heart rate, arterial tone, plethismography, and snoring levels.

3. The method of claim 1 further including determining the sleep quality index based on current physiological data.

4. The method of claim 1 including:
storing the physiological data; and
determining the sleep quality index based on historical physiological data.

5. The method of claim 1 further including determining the sleep quality index based on current physiological data and historical physiological data.

6. The method of claim 1 further including storing the sleep quality index.

7. The method of claim 1 wherein the sleep quality index is determined based on the sleep quality on current physiological data and at least one previously stored sleep quality index.

8. The method of claim 1 wherein the sleep quality index is determined based on one or more personal characteristics of the subject.

9. The method of claim 8 wherein the personal characteristics include a sleep time.

10. The method of claim 1 wherein the sleep quality index is determined based on at least one characteristic of the activity.

11. The method of claim 1 further including:
attempting to verify the identity of the subject;
if it is determined that the activity should not be permitted or the identity of the subject has not been verified, then restricting the subject from the activity; and
if it is determined that the activity should be permitted and the identity of the subject has been verified, then permitting the subject access to the activity.

12. A system for restricting an activity based on sleep quality, the system comprising:
a monitoring device adapted to monitor physiological data of a subject;
a sleep assessment device adapted to determine a sleep quality index based on the physiological data; and
an access device adapted to determine whether to permit the activity based on the sleep quality index.

13. The system of claim 12 wherein the monitoring device is adapted to monitor physiological data wherein the physiological data is selected from the group consisting of breathing patterns, electroencephalographic signals, electromyographic signals, electrooculographic signals, blood oxygen levels, body position, body movement, heart rate, arterial tone, plethismography, and snoring levels.

14. The system of claim 12 wherein the sleep assessment device is adapted to determine the sleep quality index based on current physiological data.

15. The system of claim 12 wherein the system includes a memory for storing physiological data and the sleep assessment device is adapted to determine the sleep quality index based on historical physiological data.

16. The system of claim 12 wherein the sleep assessment device is adapted to determine the sleep quality index based on current physiological data and historical physiological data.

17. The system of claim 12 wherein the sleep assessment device includes a memory for storing the sleep quality index.

18. The system of claim 17 wherein the sleep assessment device is adapted to determine the sleep quality index based on current physiological data and a previously stored sleep quality index.

19. The system of claim 12 wherein the sleep assessment device is adapted to determine the sleep quality index based on one or more personal characteristics of the subject.

20. The system of claim 19 wherein the personal characteristics include a sleep time.

21. The system of claim 12 wherein the sleep assessment device is adapted to adjust the sleep quality index based on at least one characteristic of the activity.

22. The system of claim 12 wherein the access device is adapted to verify the identity of the subject and to permit the activity based on the sleep quality index and the subject's identity.

23. The system of claim 12 further including an identification device coupled to the access device.

24. The system of claim 23 wherein the identification device is selected from a group consisting of a key, a device incorporating a RFID tag, a smart card or other memory device.
